(19) United States
(12) Patent Application Publication
(10) Pub. No.: US 2026/0137450 A1
(43) Pub. Date: May 21, 2026

(54) DIRECT IN-SCOPE SUCTION SYSTEM

(71) Applicant: BIORAD MEDISYS PRIVATE LIMITED, Pune (IN)

(72) Inventors: Debasish PRADHAN, Pune (IN); Satej Vilasrao JAGATAP, Pune (IN); Nilesh Gorakh KALE, Pune (IN)

(21) Appl. No.: 19/121,814

(22) PCT Filed: Oct. 17, 2023
(86) PCT No.: PCT/IN2023/050955

§ 371 Date: Apr. 17, 2025

(30) Foreign Application Priority Data
Oct. 17, 2022 (IN) 202221059185

(51) Int. Cl. A61B 18/26; A61B 1/00; A61B 1/307; A61B 17/00; A61B 18/00; A61B 90/00

(52) U.S. Cl. CPC A61B 18/26; A61B 1/00068; A61B 1/00128; A61B 1/0014; A61B 1/307; A61B 90/08; A61B 2017/00544; A61B 2017/00557; A61B 2017/00561; A61B 2018/00172; A61B 2018/00511; A61B 2018/00982; A61B 2090/0807; A61B 2218/002; A61B 2218/007

(57) ABSTRACT

The invention disclosed in this document presents a novel direct in-scope suction system designed for the retrieval of calculus fragments within the body. This system comprises two key components: a calculus fragments retrieval device and a pump unit. The calculus fragments retrieval device features a body with a single working channel that includes both an irrigation port and a suction port, which are connected to respective irrigation and suction pumps via elastomer-based rubber tubes. This setup allows for the controlled infusion of saline solution into the body lumens during lithotripsy procedures, ensuring clear visibility and fragment clearance. The retrieval device includes a trigger lever connected to a pincher tab. When the operator presses the trigger lever, the suction port is opened by releasing the pinched suction tube, increasing pressure inside the body.

### FIELD OF THE INVENTION

The present invention relates generally to the field of medical devices. More specifically, the present invention relates to endoscopic device with a suitable working device having an irrigation and suction system that allows easy and quick salvage of stone fragments and/or stone dust from the kidneys of a patient.

### BACKGROUND

Kidney stones (known as ureteral calculi in medical terminology) are a common medical problem that negatively impacts millions of individuals worldwide. Kidney stones include one or more solid masses of material that are usually made of crystals and form in parts of the urinary tract including in the ureter, the kidney, and/or the bladder of the individual. Kidney stones range in size from smaller (less than about 1 cm) to very large (more than 4 cm) and may cause significant pain to the individual and damage to the kidney. The overwhelming majority of stones that are treated by surgeons are less than 1 cm.

The recommended treatment for removal of the kidney stones varies according to numerous factors including the size of the kidney stones, the number of kidney stones, and the location of the kidney stones. The most common treatments for kidney stones are shock wave Lithotripsy, Ureteroscopy, Retrograde Intrarenal Surgery (RIRS) and Percutaneous Nephrolithotomy. The largest kidney stones are usually removed through Percutaneous Nephrolithotomy or Nephrolithotripsy, or through other similar procedures.

Traditionally, smaller kidney stones have been treated using other, less invasive techniques including through ureteroscopy. In ureteroscopy, the surgeon typically inserts a ureteroscope into the urethra through the bladder and the ureter to provide the surgeon with a direct visualization of the kidney stone(s) which may reside in the ureter or kidney. The surgeon then removes the kidney stone directly using a basketing device if the kidney stone is small enough to pass through the urinary tract without difficulty, or the surgeon fractures the kidney stone into smaller pieces using a laser or other breaking device. After breaking the kidney stone into smaller pieces, the surgeon removes the laser or breaking device and inserts a basket or other object to capture the kidney stone fragments under the direct visualization of the ureteroscope. Upon retrieving some of the kidney stone fragments, the surgeon removes the basket from the patient and empties the kidney stone fragments therefrom. This process is repeated until clinically significant kidney stones and kidney stone fragments are broken up and removed from the body.

Stenting is usually followed after a Laser Lithotripsy procedure under local or general anaesthesia. Patient is kept under observation and bed rest with a Foley's catheter in place to let the patient pass urine in a urine bag. After 24 hours of observation, when the urine is clear, the patient gets discharged from the hospital and is asked to keep the Stents for a week. During this time, patient is asked to collect the stone fragments through gauge or stone collector mesh. However, it is impossible to catch the stones of such small size. After one week, patient needs to get admitted again to get the ureteral stent removed.

It should be apparent that this process is extremely time consuming, costly, and inefficient because the surgeon is required to insert and remove the scope and basket into and out of the patient many times to completely remove the kidney stones and kidney stone fragments therefrom. Using a basket removal device to capture kidney stones or kidney stone fragments suffers from other drawbacks in that the basket is difficult to position adjacent the kidney stone fragments and manoeuvre in a manner that effectively retrieves the fragments. Also, most of the time the stent causes discomfort to the patient especially during movement. Patient re-admission is required for stent removal and that too under anaesthesia. Moreover, it is very difficult to catch enough stone fragments for getting a stone analysis done. It is very painful to remove Foley's Catheter as it is not done under anaesthesia. Also, it is not possible to confirm if all stone fragments gets drained out of Kidney. Additionally, the surgeon is susceptible to hand fatigue due to the extended amount of time required to operate the kidney stone retrieval baskets. Further, the patient is required to be under local anaesthesia and/or remain immobile over an extended amount of time. Still further, the basket retrieval devices cause irritation to the urinary tract due to the repeated insertion and removal therefrom.

New developments in laser technology allow for stone “dusting” rather than just fragmentation. This technique erodes stones and produces sand particles less than 0.5 mm. Intense light energy from a laser within the Ureteroscope breaks the stone into increasingly smaller pieces. Rather than breaking up the stone into chunks, which are removed by baskets, dusting generates very small fragments that are capable of being passed naturally. However, in some cases, these small stone fragments may not pass naturally. In theory, any of these small stone fragments that do not evacuate through natural urine flow, could be a seed for new stone growth Even with these recent developments, it is challenging to remove all stones and fragments, in part, because dusting creates a cloud of particles, through which remaining stones and fragments are difficult to visualize.

It would be desirable to have a more robust clearance mechanism when small stones and stone powders are present in the renal pelvis of kidney which is more difficult to access. Hence, there is an unmet demand for improvements and need for new devices and methods that permit minimally invasive removal of kidney stones.

This invention is therefore directed towards developing a system to facilitate complete removal of stone fragments or dust which cannot be captured by stone basket quickly and efficiently by application of suction.

### SUMMARY

Embodiment of the present disclosure present technological improvements as solutions to one or more of the above-mentioned technical problems recognized by the inventor(s) in conventional systems.

An object of the present invention is to provide a stone fragments retrieval system to evacuate sand and dust fragments of crushed kidney stones completely by eliminating the need for a stone capturing basket.

Another object of the present invention is to provide an improved stone fragment retrieval device and a system, more particularly to facilitate simultaneous use of a working channel for lithotripsy, irrigation and suction within the body of a patient which can be easily used by the physician.

Yet another object of the invention is to provide a stone fragments retrieval device and a system which selectively alter the suction or irrigation flow in the working channel for evacuating the sand and dust fragments of crushed kidney stones.

It is still another object of the present invention to provide a medical endoscope (ureteroscope) and a system which reduces intrarenal pressure and provide an improved visualization of stones while operating.

A further object of the invention is to avoid need for stenting and Foley's Catheter which eventually reduces surgery time thereby reducing patient recovery time.

In an aspect of the present disclosure, a direct in-scope suction system is provided which comprises of calculus fragments retrieval device and a pump unit. The calculus fragments retrieval device comprises of a body having a single working channel comprising an irrigation port and suction port connected to an irrigation and suction pump via rubber tubes made up of elastomer. The calculus fragments retrieval device also comprises of a trigger lever attached to a pincher tab. The trigger lever selectively alters the suction or irrigation flow to the working channel. In normal condition i.e. the irrigation port is normally Open and Suction port is closed, the saline liquid via peristaltic irrigation pump is infused inside the body lumens through scope as a means for clearing the stone fragments or debris as the lithotripsy progresses as well as to maintain a clear field of view through the endoscope during the procedure. When the trigger lever is pressed by the operator, the suction port gets opened by releasing the pinched suction rubber tube which increases the pressure inside the kidney. A T-connector is attached to the irrigation and suction unit comprising a diaphragm that gets inflated due to increased pressure which eventually activates a switch enabling it to give signals to PCB to close the peristaltic irrigation pump. The stone dust along with the liquid inside the body lumens gets extracted and pass through the filter into the dust collection canister. Once the suction function is performed and all the dust fragments are extracted, the operator can release the trigger lever back to its original position.

Additional aspects, advantages, features and objects of the present disclosure would be made apparent from the drawings and the detailed description of the illustrative embodiment.

It will be appreciated that features of the present disclosure are susceptible to being combined in various combinations without departing from the scope of the present disclosure as defined by the below mentioned detailed description and drawings.

### BRIEF DESCRIPTION OF THE FIGURES

The summary above, as well as the following detailed description of illustrative embodiment, is better understood when read in conjunction with the appended drawings. For illustrating the present disclosure, example constructions of the disclosure are shown in the drawings. However, the present disclosure is not limited to specific methods and instrumentalities disclosed herein. Moreover, those in art will understand that the drawings are not to scale. Wherever possible, like elements have been indicated by identical numbers.

Embodiment of the present disclosure will now be described, by way of example only, with reference to the following diagrams wherein:

FIG. 1. illustrates an isometric view of the direct in-scope suction system in accordance with an exemplary embodiment of the present disclosure.

FIG. 2. illustrates an isometric view of the calculus fragments retrieval device attached to an endoscope in accordance with an exemplary embodiment of the present disclosure;

FIG. 3 illustrates an exploded view of the calculus fragments retrieval device with an exemplary embodiment of the present disclosure;

FIG. 4(a)-4(b) illustrates a sectional view of the calculus fragments retrieval device in accordance with an exemplary embodiment of the present disclosure;

FIG. 5 illustrates a filter device in accordance with an exemplary embodiment of the present disclosure;

FIG. 6(a) illustrates an exploded view of the pump unit of direct in-scope suction system in accordance with an exemplary embodiment of the present disclosure;

FIG. 6(b) illustrates a top view of the pump unit of the direct in-scope suction system in accordance with an exemplary embodiment of the present disclosure;

FIG. 6(c) illustrates a perspective view of tubing assembly of the pump unit of the direct in-scope suction system in accordance with an exemplary embodiment of the present disclosure;

FIG. 7 illustrates a schematic view of the direct in-scope suction system in accordance with an exemplary embodiment of the present disclosure.

In the above accompanying drawings, a number relates to an item identified by a line linking the number to the item. When a number is accompanied by an associated arrow, the number is used to identify a general item at which the arrow is pointing.

Further the figures depict various embodiments of the present subject matter for purposes of illustration only. One skilled in the art will readily recognize from the following discussion that alternative embodiment of the structures and methods illustrated herein may be employed without departing from the principles of the present subject matter described herein.

### DETAILED DESCRIPTION OF EMBODIMENTS

The following detailed description illustrates embodiment of the present disclosure and manners by which they can be implemented. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise. As used herein, a “plurality” refers to two or more, for example, three or more, four or more, five or more, six or more. Each possibility represents a separate embodiment of the invention.

For purposes of the present specification and claims, various relational terms like “top” “bottom,” “proximal,” “distal,” “upper,” “lower,” “front,” and “rear” are used to describe the present invention when said invention is positioned in or viewed from a given orientation. It is to be understood that, by altering the orientation of the invention, certain relational terms may need to be adjusted accordingly.

The person skilled in the art will recognize many variations, alternatives, and modifications of the embodiment of the present disclosure. It should be understood that this invention is not limited to the particular methodology, protocols, and the like, described herein and as such may vary. The terminology used herein is for the purpose of describing particular embodiment only and is not intended to limit the scope of the present invention, which is defined solely by the claims.

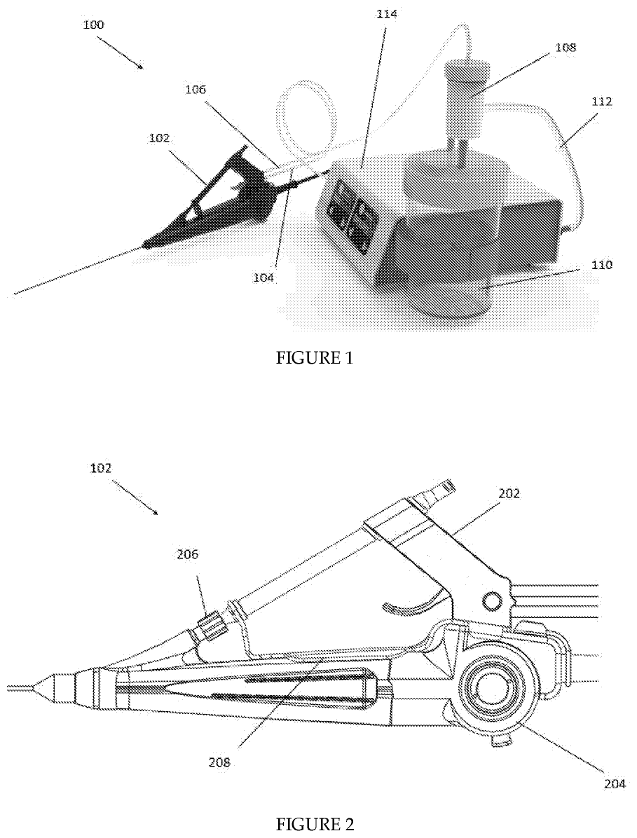

Referring now to the drawings, FIGS. 1-7 illustrates a direct in-scope suction system having an improved suction and irrigation mechanism to completely extract the dust fragments from the kidney in a more efficient and quick manner according to present invention. It should be noted that FIG. 1-7 are merely examples. A person skilled in the art will recognize many variations, alternatives, and modifications of the embodiments of the present disclosure.

Referring now to the drawings, FIG. 1 illustrates a direct in-scope suction system 100 comprising a calculus fragment retrieval device 102 comprising a plurality of tubes in particular an irrigation tube 104 and a suction tube 106. The suction tube is connected to a filter 108 to collect the dust fragments/sand which is in fluid communication with a canister 110 to collect the water. The outlet tube 112 from the canister 110 is connected to an irrigation suction pump unit 114.

FIG. 2 illustrates a calculus fragments retrieval device 102 comprising a body 202 configured to be coupled to an endoscope 204 with the help of a scope port connector 206 and a flange 208 which is to be seated on the endoscope 204. The scope port connector 206 is adapted to be attached with 7.5 Fr or 9 Fr Flexible Ureteroscope.

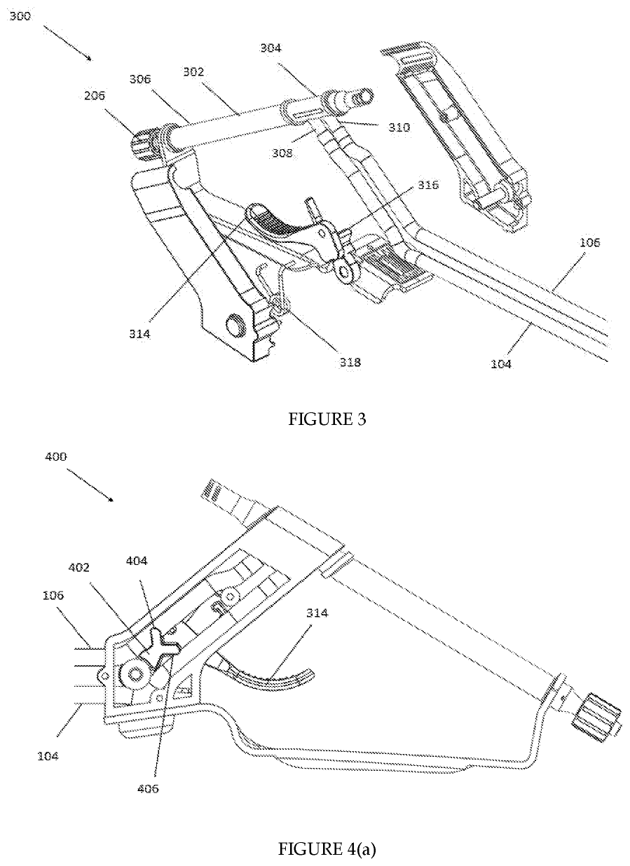

Now referring to FIG. 3, the exploded view 300 of the calculus fragments retrieval device 102 is illustrated. The body 202 comprises of a long section 302 (hereafter referred as a “working channel”) having a proximal end 304 and a distal end 306. The distal end 306 is coupled to the endoscope 204 with the help of a scope port connector 206. In the laser lithotripsy procedure, the kidney stones are disrupted and broken up using laser which generate dust particles within the body cavity. A laser fibre is introduced through the proximal end 304 of the working channel 302 and the procedure of laser lithotripsy is performed.

An irrigation port 308 and a suction port 310 are provided near the proximal end 304 of the working channel 302 of the body 202 to provide saline to the urinary system and to extract the dust fragments. Both ports i.e., irrigation port 308 and suction port 310 are connected to the working channel 302 through an irrigation rubber tube 104 and a suction rubber tube 106 in the body 202 which are further connected to the irrigation suction pump unit 114 as shown in FIG. 1. The rubber tubes 104,106 are made up of elastomer materials like silicon and the like. The body 202 also comprises of a trigger lever 314 which is attached to the pincher tab 316. The trigger lever 314 can rotate certain degrees against a torsion spring 318 coupled to the body 202. The spring 318 helps the trigger lever 314 to return to fixed original position after rotating a certain degree. The calculus retrieval device is made up of plastic material.

FIG. 3 is merely an example. A person skilled in the art will recognize many variations, alternatives, and modifications of the embodiment of the present disclosure. The material of the tubes in the present invention is not limited to the elastomers but can be different according to the requirement of the thickness and flexibility of the tube.

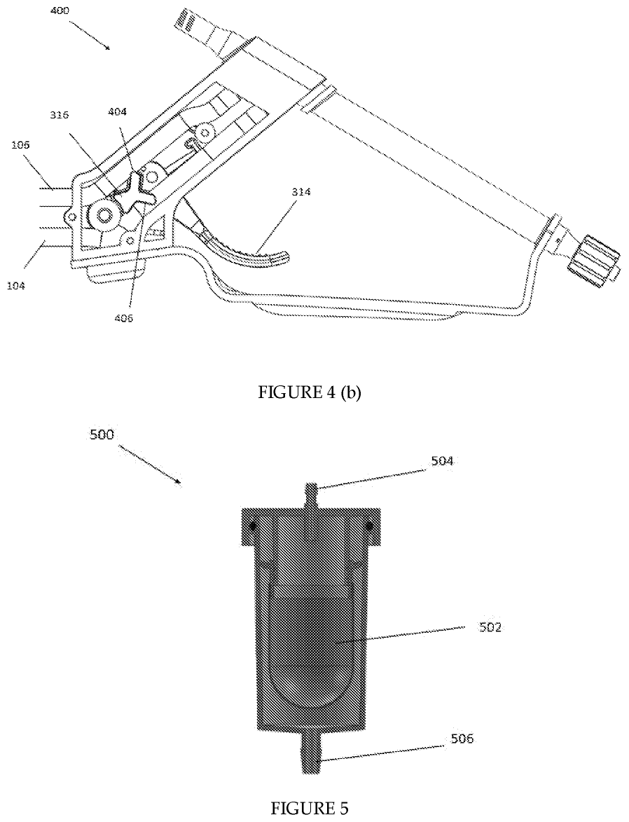

Referring now to FIG. 4(a)-4(b), the sectional view 400 of the of the calculus fragment retrieval device body 202 is disclosed. FIG. 4(a) shows the calculus fragment retrieval device 102 in a normal condition i.e. the irrigation port 308 is open and suction port 310 is closed. While performing an endoscopic procedure with a stone retrieval device within a body cavity or passageway, and especially while performing lithotripsy, a common problem is that stone fragments or debris from the lithotripsy clouds the endoscope's field of view. A means for clearing such debris is desirable both to remove the debris as the lithotripsy progresses, and to maintain a clear field of view through the endoscope during the procedure. During Irrigation function, saline liquid is infused inside the body lumens through scope. The saline water is required for maintaining clear visibility inside the body and is also required for dusting operation as medium of power transfer for laser. The pincher tab 402 has an upper tab 404 and a lower tab 406. As shown in FIG. 4(a), the suction rubber tube 106 is pinched using the upper pincher tab 404 attached to the trigger lever 314. The suction rubber tube 106 is pinched between the wedge of the pincher tab 402 and wall of the body 202. The torsion spring 318 keeps the trigger lever 314 in a position where the suction rubber tube 106 is always pinched.

The irrigation and suction rubber tubes 104,106 respectively are arranged in such a way that only single rubber tube can be made operational at once. The trigger lever 314 is used to select between either of the functions of suction or irrigation. Suction function is performed to extract the stone dust fragments along with the liquid inside the body lumens. FIG. 4(b) illustrates the calculus fragment retrieval device when the suction port 310 is open and the irrigation port 308 is closed. The lower tab 406 pinches the irrigation rubber tube 104 while the operator presses the trigger lever 314 thereby releasing the suction rubber tube 106 from the pinched state, opening the suction port 310 and evacuating the stone dust fragments and sand from the suction rubber tube 106 via suction port 310.

FIG. 4(a)-4(b) are merely examples. A person skilled in the art will recognize many variations, alternatives, and modifications of the embodiment of the present disclosure.

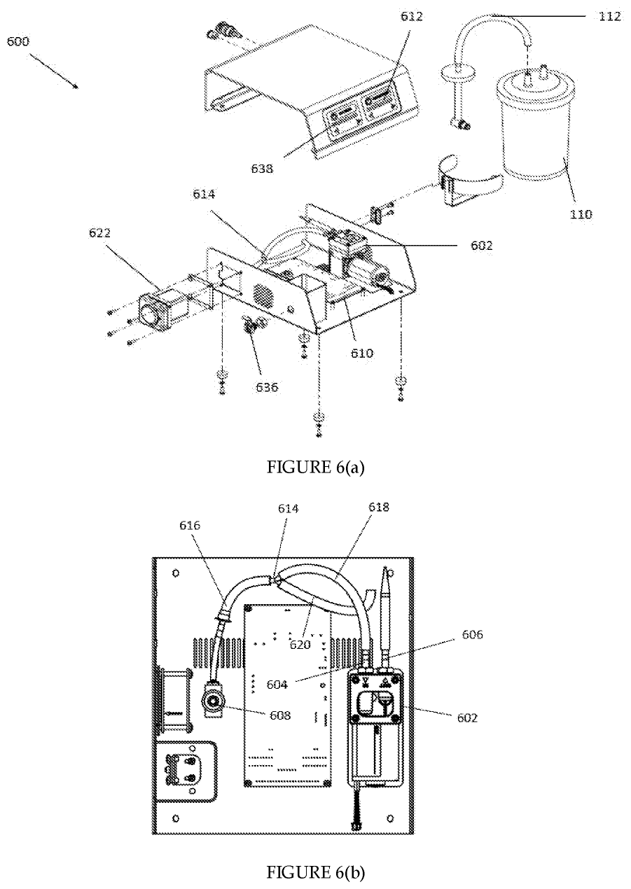

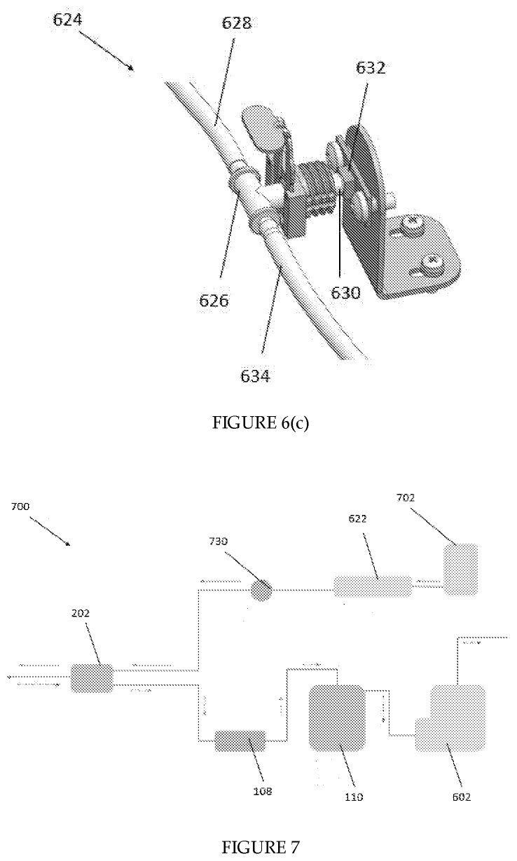

FIG. 5 illustrates the cross section 500 of the filter 108 connected to a collector canister 110 to collect the urine and water. The filter 108 is configured as a chamber and may include one or more particulate filters to allow quantification and collection of sand and debris. The filter 108 and canister 110 is adapted to be in fluid communication with both calculus fragment retrieval device and pump unit 114 via suction tube 106 and may be positioned downstream from ureteroscope 204 and upstream of irrigation suction pump unit 114. During operation, when the suction port 310 is open the filter 108 captures the dust contaminants of big size i.e. coarse dust is captured by the media 502 of the filter 108 when the urine and dust fragments enters the filter 108 through an inlet 504 of the filter 108. The small size contaminants in the form of fine dust are allowed to be passed by the media 502 of the filter 108 through the outlet 506 of the filter 108 and the mixture of fine dust and urine gets collected in the collection container 110. The outlet of the drain collection canister 110 is connected to the suction pump 602 as shown in FIG. 6(a). The collected sample of the dust in the filter can be given directly to the pathology lab for further testing.

FIG. 6(a) illustrates an exploded view 600 of the pump unit 114. The irrigation suction pump unit 114 comprises of suction pump 602 having an inlet 604 and outlet port 606. The pump unit 114 further comprises of a proportional valve 608 with PCB 610 (shown in FIG. 7(b)). The proportional valve 608 regulates the vacuum that is been created in the suction canister 114 with the help of a suction keypad 612 provided on the pump unit 114 by passing variable current to the proportional valve 608. The user presses the suction keypad 612 which in turn opens and closes the proportional valve 608. If the user increases the reading on the keypad 612, the opening in proportional valve 608 starts getting close which in turn increases the suction. If the opening in proportional valve 608 is fully closed, the suction is maximum and vice versa.

Furthermore, a Y-tube assembly 614 wherein a common tube 616 is connected to the proportional valve 608, a first tube 618 is connected to the inlet port 604 of the suction pump 602 and a second tube 620 is connected to the suction canister tube 112.

A saline solution is continuously fed to a peristaltic irrigation pump 622. The pump unit 114 further comprises of an irrigation tube assembly 624 wherein 3 tubes are connected via a T-connector 626. One portion of plastic T-connector 626 is connected to a peristaltic irrigation pump 622 via a tube 628 (as shown in FIG. 7(b)) that is further connected to the saline solution. A middle portion of T-connector 626 extends inside the irrigation suction pump unit 114 adapted to be covered by a diaphragm/a pressure sensor device/or a switch actuator 630 positioned inside the irrigation suction pump unit 114 in a proximity of a switch 632. Third end of T-connector 626 is connected to the calculus fragment retrieval device body 202 via a tube 634 which continuously feed saline solution to the kidneys via working channel during operation. The T-connector 626 is mounted on the switch actuator holder assembly 636 that is mounted on an outer periphery of irrigation suction pump unit 114. The calculus fragment retrieval device 102, filter canister 108 and the T-connector assembly 626 are disposable and needs to be replaced after every surgery.

FIG. 7 illustrates a schematic diagram 700 of the direct in scope suction system 100. As can be seen, saline solution 702 is fed to a peristaltic irrigation pump 622. In a default mode, the suction port 310 is closed. When the physician presses the trigger lever 314, the irrigation port 308 is closed and suction port 310 is open which builds the pressure at the irrigation pump 622 which may be harmful to the patient. The rubber diaphragm 630 at the second end of T-connector 626 inflates due to this increased pressure which activates the tactile switch 632 to feed signals to a PCB 610 in the irrigation suction pump unit 114 to switch off the irrigation pump 622 in the form of LED on an irrigation keypad 638. Sand and dust fragments are evacuated out of the kidneys to the collection container 110 via suction port 310, suction tube 106 and filter 108.

It is observed that the dust fragments after lithotripsy procedure can be extracted out more quickly and efficiently.

It is further observed that along with the elimination of the stone capturing basket means, the direct in-scope suction system has provided the much-needed advantage of extracting the dust fragments from the kidneys efficiently which avoids any further discomfort to the patient as it was the case in conventional methods of removing the stones from the kidneys. Along with these advantages, there is a quick and complete extraction of dust fragments, eventually saving the time of the patient as well as the physician described in the present invention.

The present invention has both technical as well as economic significance with respect to the conventional kidney stone retrieval devices.

While a particular embodiment of the invention has been illustrated and described, modifications thereof will readily occur to those skilled in the art. It is understood that the various embodiment, details and constructions of the calculus fragments retrieval device, direct in-scope suction system and their features described above and illustrated in the attached figures may be interchanged among the various embodiment while remaining within the scope of the invention. Additionally, it is understood that various modifications could be made to any of the calculus fragments aspiration device and/or elements described herein above while remaining within the scope of the invention.

We claim:

1. A direct in scope suction system for removing calculus fragments from a patient comprising:
A calculus fragment retrieval device 102 comprising a body 202 having a channel 302 comprising a proximal end 304 and a distal end 306 configured to be removably attached to an ureteroscope 104 via a scope connecting port 206,
wherein:
the said channel 302 acts as a medium to introduce laser fibre or any other accessory for surgery, infusing saline liquid to kidneys as well as to aspire debris from the kidneys of a patient;
the said channel 302 further comprising an inlet port 308 and an outlet port 310 near the proximal end 304 of the said channel 302,
wherein:
the said inlet port 308 and the outlet port 310 are connected to an inlet tube 104 and an outlet tube 106, both of which are further connected to a pump unit 114 for irrigation and negative pressure; and
a trigger lever 314 being rotatable against a torsion spring 318 and is connected to a pincher tab 316 on the said body 102,
wherein:
the said pincher tab 316 comprises of an upper tab 404 and a lower tab 406 that selectively controls the suction and irrigation flow through the working channel when the user presses the trigger lever 314.

2. The calculus fragment retrieval device as claimed in claim 1, wherein the inlet port 308 is an irrigation port to infuse saline liquid into the kidneys of a patient and is connected to an irrigation supply source via inlet tube 104.

3. The calculus fragment retrieval device as claimed in claim 1, wherein the outlet port 310 is a suction port to aspire debris and urine from the kidneys of a patient and is connected to a negative supply source via outlet tube 106.

4. The calculus fragment retrieval device as claimed in claim 1, wherein when the trigger lever 314 is in normal position the upper tab 404 of the pincher 316 keeps the flow from the suction tube 106 blocked for the continuous flow of saline liquid through the irrigation tube 104.

5. The calculus fragment retrieval device as claimed in claim 1, wherein when the user presses trigger lever 314, the negative pressure gets applied and the lower tab 406 of the pincher 316 blocks the flow from the irrigation tube 104 allowing the debris and urine to eventually get removed through the suction tube 106.

6. A direct in scope suction system for removing calculus fragments from a patient comprising:
a calculus fragment retrieval device 102 and a pump unit 114, wherein:
the pump unit 114 comprises a suction pump 602 to supply negative pressure to the calculus fragment retrieval device body;
a proportional valve 608 connected to a Printed Circuit Board 610, wherein:
the said proportional valve 608 regulates the vacuum that is been created in a suction canister 110 attached to the said pump unit 114;
a peristaltic irrigation pump 622 to supply saline solution to the calculus fragment retrieval device body via an irrigation tube assembly 624; and
a diaphragm 630 positioned inside the said irrigation suction pump unit 114 in a proximity of a switch 632, wherein:
when the trigger lever 314 is pressed by an operator, the suction port 310 is opened by releasing the pinched suction tube 106, which increases the pressure inside the body lumen, and the diaphragm 630 is inflated due to the increased pressure, which activates the switch 632 and signals to a PCB 610 to close the irrigation pump 622.

7. The direct in scope suction system as claimed in claim 6, wherein, the said irrigation tube assembly 624 comprises of 3 tubes connected via a T-connector 626. One portion of plastic T-connector 626 is connected to a peristaltic irrigation pump 622 via a tube 628 that is further connected to the saline solution. A middle portion of T-connector 626 extends inside the irrigation suction pump unit 114 adapted to be covered by a diaphragm 630 positioned inside the irrigation suction pump unit 114 in a proximity of a switch 632. Third end of T-connector 626 is connected to the calculus fragment retrieval device body 202 via a tube 634 which continuously feed saline solution to the kidneys via working channel during operation.

8. The direct in scope suction system as claimed in claim 6, wherein the proportional valve 608 regulates the vacuum being created in the suction canister 114 with the help of a suction keypad 612 provided on the pump unit 114 by passing variable current to the proportional valve 608.

9. The direct in-scope suction system of claim 6, the closure of the peristaltic irrigation pump due to increased pressure is given in the form of LED on an irrigation keypad 638.
